(19) United States
(12) Patent Application Publication
(10) Pub. No.: US 2019/0076431 A1
(43) Pub. Date: Mar. 14, 2019

(54) SRC INHIBITOR TO BLOCK CELL SURFACE GRP78 EXPRESSION

(71) Applicant: University of Southern California, Los Angeles, CA (US)

(72) Inventors: Amy S. Lee, Los Angeles, CA (US); Yuan-li Tsai, Los Angeles, CA (US)

(21) Appl. No.: 16/085,229

(22) PCT Filed: Mar. 16, 2017
(86) PCT No.: PCT/US2017/022834

§ 371 Date: Sep. 14, 2018

Related U.S. Application Data
(60) Provisional application No. 62/309,561, filed on Mar. 17, 2016.

(51) Int. Cl. A61K 31/517; A61P 35/00; A61K 31/496; A61K 38/21; A61P 31/12; A61P 31/10

(52) U.S. Cl. CPC A61K 31/517; A61P 35/00; A61K 31/496; A61P 31/10; A61K 38/212; A61K 38/217; A61P 31/12; A61K 38/215

(57) ABSTRACT

Methods of treating cancer, tumor angiogenesis, viral infection and fungal entry are provided. These methods include administering to a subject in need thereof an effective amount of a pharmaceutical composition that includes a compound that inhibits pro-to-oncogene tyrosine kinase protein SRC (c-SRC). The inhibition of SRC blocks cell surface glucose-regulated protein GRP78 and/or other endoplasmic reticulum ER luminal proteins dependent on SRC from going to the cell surface.

### RELATED APPLICATIONS

This application claims the benefit of U.S. Provisional Application No. 62/309,561, filed Mar. 17, 2016, the entire contents of which are incorporated herein by reference in its entirety.

### STATEMENT REGARDING FEDERALLY SPONSORED RESEARCH OR DEVELOPMENT

This invention was made with government support under National Institutes of Health/National Center for Biotechnology Information grant CA R01-027607. The government has certain rights in the invention.

### FIELD OF THE INVENTION

The present invention relates to the blocking of cell surface glucose-regulated protein (GRP78) expression in the treatment of cancer and the prevention of fungal or viral entry into host cells.

### BACKGROUND OF THE INVENTION

The glucose regulated protein, GRP78 (also referred to as BiP or HSPAS), is a member of the heat shock protein 70 (HSP70) superfamily and evolutionarily conserved from yeast to human (1, 2). GRP78 contains a signal peptide that targets it to endoplasmic reticulum (ER) and a carboxyl KDEL motif for retrieval from the Golgi apparatus leading to ER retention (3). The ER is an essential organelle for the synthesis and processing of plasma membrane and secretory proteins. As a major ER chaperone protein with ATPase activity, GRP78 complexes with nascent polypeptides and is critical for their folding and maturation in the ER compartment. Under ER stress conditions, when malfolded proteins accumulate in the ER, GRP78 is up-regulated and prevents protein aggregation as well as facilitates degradation of misfolded proteins (1, 4). GRP78 is a key regulator of the unfolded protein response (UPR) such that it binds and maintains the transmembrane ER stress sensors (PERK, IRE1 and ATF6) in their inactive forms, and upon ER stress, GRP78 is released resulting in the activation of these signaling pathways, impacting both cell survival and apoptosis (4, 5). Analogously, in non-stressed cells, GRP78 forms complex with ER-associated pro-apoptotic signaling machineries and blocks their activation (2).

While traditionally GRP78 has been regarded as an ER lumenal protein, evidence is emerging that GRP78 can also be detected in other cellular locations including the cell surface, cytosol, mitochondria and the nucleus, and assume novel functions that control signaling, proliferation, invasion, apoptosis, inflammation and immunity (2, 4, 6). Of particular importance is that a subfraction of GRP78 can relocalize to the surface of specific cell types, such as cancer cells, and this process is actively enhanced by ER stress (7-9). At the cell surface, in complexes with specific cell surface proteins, GRP78 exerts functions beyond the ER (6). For example, GRP78 serves as co-receptor for the proteinase inhibitor α2-macroglobulin induced signal transduction for cancer survival and metastasis (10). Cell surface GRP78 is also an obligatory binding partner for Cripto, a GPI-anchored protein on the cell surface for its activity in regulating stem cell regeneration and tumorigenesis (11, 12). Cell surface GRP78 can also mediate endothelial cell survival, TRAIL-induced apoptosis as well as viral entry into host cells. The recent discovery that GRP78 is preferably expressed on the surface of tumor cells but not normal organs in vivo opens a unique opportunity for specific tumor targeting with minimal harmful effects on normal cells. As cell surface GRP78 is further detected in some tumor initiating cells and increased in metastatic and cancer cells that have developed therapy resistance, as well as in hypoxic endothelial cells that support tumor cells, cytotoxic agents including peptide-drug conjugates and monoclonal antibodies targeting against cell surface GRP78 has shown great promise in cancer therapy in multiple settings and are currently under development (2, 7, 8, 13-18).

Japanese encephalitis virus (JEV), a mosquito-borne flavivirus, is the leading cause of viral encephalitis in Southeast Asia with potential to become a global pathogen. A paper published after the filing U.S. Provisional Application No. 62/309,561 (the application from which the present application claims priority) identified cell surface glucose-regulated protein 78 (GRP78) as an important host protein for virus entry and replication (69). Using the plasma membrane fractions from mouse neuronal (Neuro2a) cells, mass spectroscopy analysis identified GRP78 as a protein interacting with recombinant JEV envelope protein domain III. GRP78 was found to be expressed on the plasma membranes of Neuro2a cells, mouse primary neurons, and human epithelial Huh-7 cells. Antibodies against GRP78 significantly inhibited JEV entry in all three cell types, suggesting an important role of the protein in virus entry. Depletion of GRP78 by small interfering RNA (siRNA) significantly blocked JEV entry into Neuro2a cells, further supporting its role in virus uptake. Immunofluorescence studies showed extensive colocalization of GRP78 with JEV envelope protein in virus-infected cells. This interaction was also confirmed by immunoprecipitation studies. Additionally, GRP78 was shown to have an important role in JEV replication, as treatment of cells post-virus entry with subtilase cytotoxin that specifically cleaved GRP78 led to a substantial reduction in viral RNA replication and protein synthesis, resulting in significantly reduced extracellular virus titers. These results indicated that GRP78, an endoplasmic reticulum chaperon of the HSP70 family, which can also translocate to the cell surface, is a host factor involved at multiple steps of the JEV life cycle.

Considering the significance of cell surface GRP78 from both the basic cell biology and therapeutic targeting perspective, it is important to understand how GRP78 exists stably on the cell surface and how it reaches the cell surface. This is particularly intriguing since the primary amino acid sequence of the mature GRP78 contains only a few weak hydrophobic domains, and GRP78 containing the intact KDEL ER retrieval motif is capable of localizing on the cell surface (9, 15). Global profiling of cell surface proteome of tumor cells clearly revealed relative abundance of cytosolic heat shock and ER lumen chaperones, including GRP78 (19), suggesting relocating these stress-inducible chaperones to the cell surface could represent a common adaptive mechanism for cells to respond to stress perturbing protein homeostasis.

### SUMMARY OF THE INVENTION

One aspect of the present invention is directed to a method of treating cancer. The method includes administering to a subject in need thereof an effective amount of a pharmaceutical composition that includes a compound that inhibits proto-oncogene tyrosine kinase protein SRC (c-SRC). The inhibition of SRC blocks cell surface glucose-regulated protein GRP78 and/or other endoplasmic reticulum ER luminal proteins dependent on SRC from going to the cell surface.

In one embodiment, the compound that inhibits SRC includes one or more of the following compounds: dasatinib, saracatinib, bosutinib and KX-01.

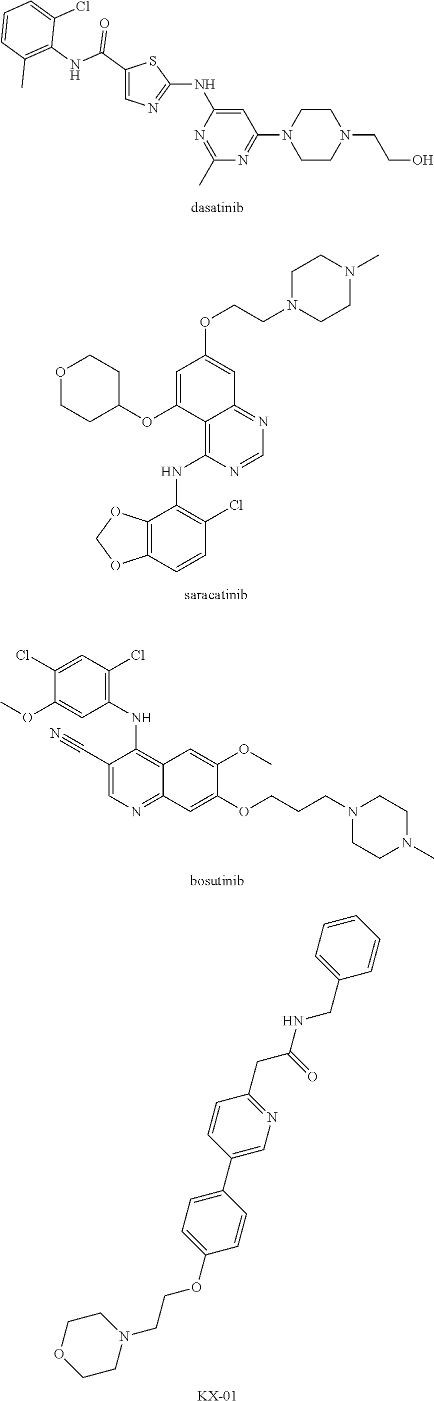

In another embodiment, the compound that inhibits SRC is dasatinib.

In another embodiment, the cancer includes bladder cancer, breast cancer, ovarian cancer, pancreatic cancer, and gastric cancer, cervical cancer, colon cancer, endometrial cancer, head and neck cancer, lung cancer, melanoma, multiple myeloma, leukemia, non-hodgkin's lymphoma, prostate cancer, rectal cancer, malignant melanomas, alimentary/gastrointestinal tract cancer, liver cancer, skin cancer, lymphoma, kidney cancer, muscle cancer, bone cancer, brain cancer, eye or ocular cancer, rectal cancer, colon cancer, cervical cancer, bladder cancer, oral cancer, benign and malignant tumors, stomach cancer, corpus uteri, testicular cancer, renal cancer, throat cancer, acute lymphocytic leukemia, acute myelogenous leukemia, Ewing's Sarcoma, Kaposi's Sarcoma, basal cell carinoma and squamous cell carcinoma, small cell lung cancer chorincarcinoma, rhabdomyosarcoma, angiosarcoma, hemangioendothelioma, Wilms Tumor, neuroblastoma, mouth/pharynx cancer, esophageal cancer, larynx cancer, neurofibromatosis, tuberous sclerosis, hemangiomas, and lymphangiogenesis.

In another embodiment, the method includes concurrently or sequentially administering to the subject one or more additional treatments that include chemotherapy, immune and radiation therapy.

In another embodiment, the pharmaceutical composition includes a therapeutic antibody, an antibody-drug conjugate, a radioimmunotherapy agent, a small molecule therapeutic agent or an immune stimulating agent.

In another embodiment, the cancer is multiple myeloma.

Another aspect of the present invention is directed to a method of treating multiple myeloma. The method includes administering to a subject in need thereof an effective amount of dasatinib.

Another aspect of the present invention is directed to a method of hindering or preventing tumor angiogenesis. The method includes administering to a subject in need thereof an effective amount of a pharmaceutical composition that includes a compound that inhibits proto-oncogene tyrosine kinase protein SRC (c-SRC). The inhibition of SRC blocks cell surface glucose-regulated protein GRP78 and/or other endoplasmic reticulum ER luminal proteins dependent on SRC from going to the cell surface.

In one embodiment, the compound that inhibits SRC includes one or more of the following compounds: dasatinib, saracatinib, bosutinib and KX-01.

In another embodiment, the compound that inhibits SRC is dasatinib.

In another embodiment, the pharmaceutical composition includes a therapeutic antibody, an antibody-drug conjugate, a radioimmunotherapy agent, a small molecule therapeutic agent or an immune stimulating agent.

Another aspect of the present invention is directed to a method of hindering or preventing viral infection into human cells. The method includes administering to a subject in need thereof an effective amount of a pharmaceutical composition that includes a compound that inhibits proto-oncogene tyrosine kinase protein SRC (c-SRC). The inhibition of SRC blocks cell surface glucose-regulated protein GRP78 and/or other endoplasmic reticulum ER luminal proteins denendent on SRC from going to the cell surface.

In one embodiment, the compound that inhibits SRC includes one or more of the following compounds: dasatinib, saracatinib, bosutinib and KX-01.

In another embodiment, the compound that inhibits SRC is dasatinib.

In another embodiment, the pharmaceutical composition includes a therapeutic antibody, an antibody-drug conjugate, a radioimmunotherapy agent, a small molecule therapeutic agent or an immune stimulating agent.

Another aspect of the present invention is directed to a method of hindering or preventing fungal entry into host cells. The method includes administering to a subject in need thereof an effective amount of a pharmaceutical composition that includes a compound that inhibits proto-oncogene tyrosine kinase protein SRC (c-SRC). The inhibition of SRC blocks cell surface glucose-regulated protein GRP78 and/or other endoplasmic reticulum ER luminal proteins dependent on SRC from going to the cell surface.

In one embodiment, the compound that inhibits SRC includes one or more of the following compounds: dasatinib, saracatinib, bosutinib and KX-01.

In another embodiment, the compound that inhibits SRC is dasatinib.

In another embodiment, the pharmaceutical composition includes a therapeutic antibody, an antibody-drug conjugate, a radioimmunotherapy agent, a small molecule therapeutic agent or an immune stimulating agent.

Another aspect of the present invention is directed to a method of hindering or preventing Listeria monocytogenes infection into human cells. The method includes administering to a subject in need thereof an effective amount of a pharmaceutical composition that includes a compound that inhibits proto-oncogene tyrosine kinase protein SRC (c-SRC). The inhibition of SRC blocks cell surface glucose-regulated protein GRP78 and/or other endoplasmic reticulum ER luminal proteins dependent on SRC from going to the cell surface.

In one embodiment, the compound that inhibits SRC includes one or more of the following compounds: dasatinib, saracatinib, bosutinib and KX-01.

In another embodiment, the compound that inhibits SRC is dasatinib.

In another embodiment, the pharmaceutical composition includes a therapeutic antibody, an antibody-drug conjugate, a radioimmunotherapy agent, a small molecule therapeutic agent or an immune stimulating agent.

Another aspect of the present invention is directed to a method of reducing drug resistance in cancer cells. The method includes administering to a subject in need thereof an effective amount of a pharmaceutical composition that includes a compound that inhibits proto-oncogene tyrosine kinase protein SRC (c-SRC). The inhibition of SRC blocks cell surface glucose-regulated protein GRP78 and/or other endoplasmic reticulum ER luminal proteins dependent on SRC from going to the cell surface.

In one embodiment, the compound that inhibits SRC includes one or more of the following compounds: dasatinib, saracatinib, bosutinib and KX-01.

In another embodiment, the compound that inhibits SRC is dasatinib.

In another embodiment, the pharmaceutical composition includes a therapeutic antibody, an antibody-drug conjugate, a radioimmunotherapy agent, a small molecule therapeutic agent or an immune stimulating agent.

Another aspect of the present invention is directed to a method of hindering or preventing viral entry into host cells. The method includes administering to a subject in need thereof an effective amount of a pharmaceutical composition that includes a compound that inhibits proto-oncogene tyrosine kinase protein SRC (c-SRC). The inhibition of SRC blocks cell surface glucose-regulated protein GRP78 and/or other endoplasmic reticulum ER luminal proteins dependent on SRC from going to the cell surface.

In one embodiment, the compound that inhibits SRC includes one or more of the following compounds: dasatinib, saracatinib, bosutinib and KX-01.

In another embodiment, the compound that inhibits SRC is dasatinib.

In another embodiment, the pharmaceutical composition includes a therapeutic antibody, an antibody-drug conjugate, a radioimmunotherapy agent, a small molecule therapeutic agent or an immune stimulating agent.

Other aspects and advantages of the invention will be apparent from the following description and the appended claims.

### BRIEF DESCRIPTION OF THE DRAWINGS

FIG. 1. Functions of Cell Surface GRP78. GRP78 normally resides in the endoplasmic reticulum (ER) but a subfraction can relocalize to the cell surface. Cell surface GRP78 is upregulated in stressed cells in vivo but not in normal organs. GRP78 controls multiple functions at the cell surface including proliferation, apoptosis, survival, viral entry. Blocking the translocation of GRP78 from the endoplasmic reticulum (ER) to the cell surface will suppresses the above cellular functions. Cell surface GRP78 forms complexes with a variety of extracellular ligands (e.g., activated α₂-macroglobulin, Kringle 5, Par-4) and cell surface anchored (▾) proteins (e.g., Cripto, T-cadherin) in tumor and endothelial cells leading to pro-survival or pro-apoptotic pathways. It also regulates the coagulation cascade through interaction with integral membrane protein (Tissue factor) and facilitates fungal (R. oryzae) and viral entries (e.g., Coxsackie virus A9, Borna disease virus and dengue virus serotype 2) in the respective host cells. 78: GRP78; α₂M*: activated α₂-macroglobulin.

FIG. 2. A C-terminal Signal Prevents Secretion of Endoplasmic Proteins.

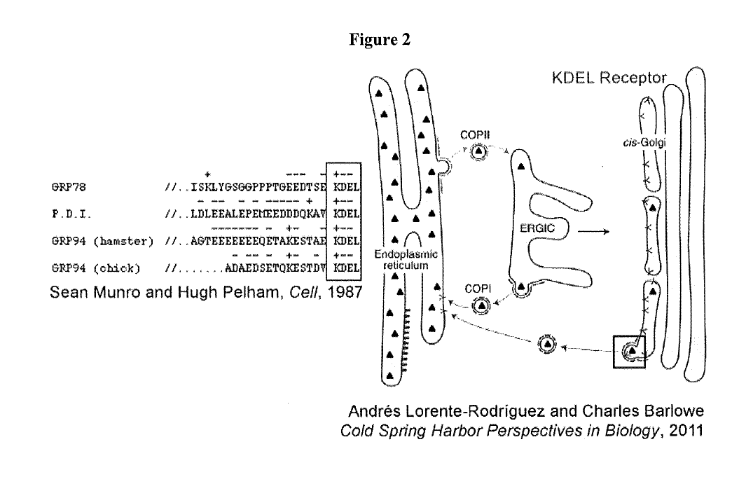

FIG. 3. SRC is Activated by ER Stress.

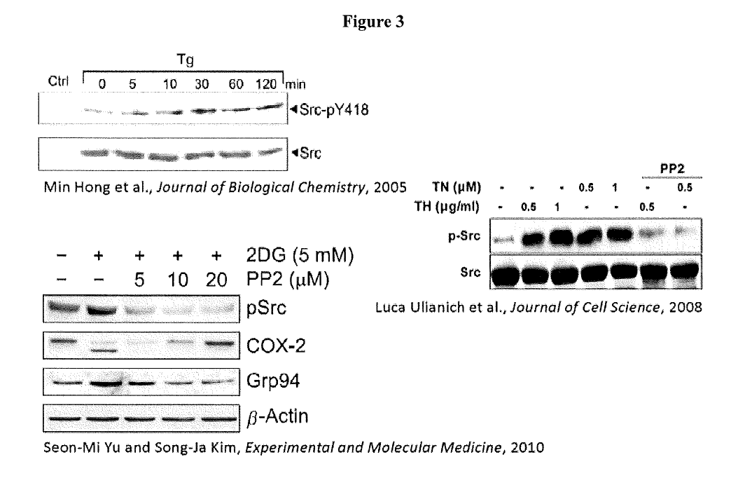

FIG. 4. Src activation correlates with cell surface expression of ER luminal KDEL-containing proteins, but not EphB4, a cell surface protein known to traffic through ER-Golgi anterograde, suggesting Golgi-ER retrograde is affected.

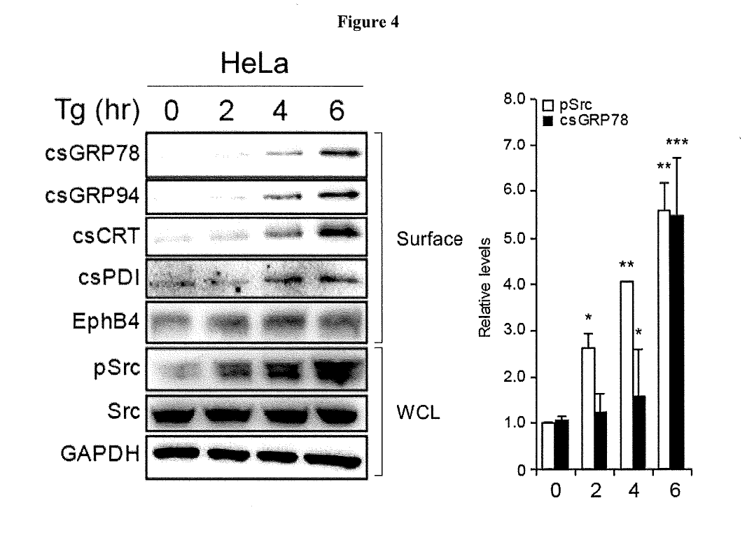

FIG. 5. SRC Kinase Activity is Essential for ER Stress-Induced Cell Surface GRPs Expression.

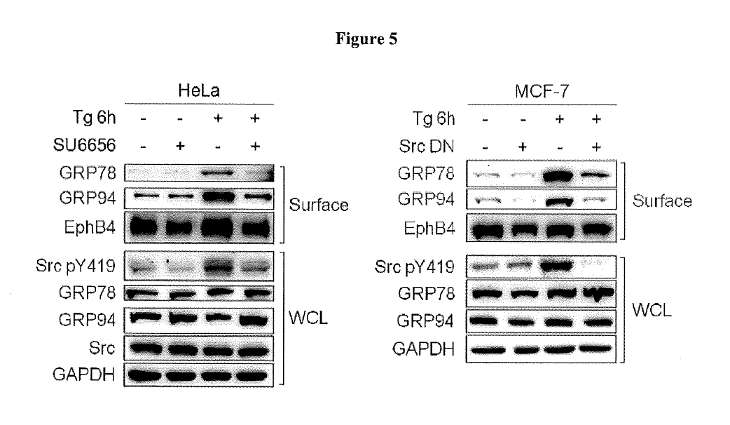

FIG. 6. SRC, not other SRC Family Kinases, is Required for ER Stress-Induced Cell Surface GRPs Expression.

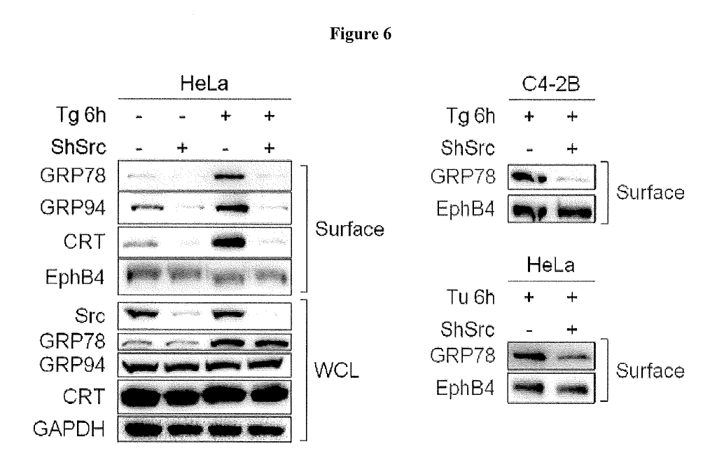

FIG. 7. Active Promotion of GRP78 to the Cell Surface in Cancer Cells Resistant to Therapy.

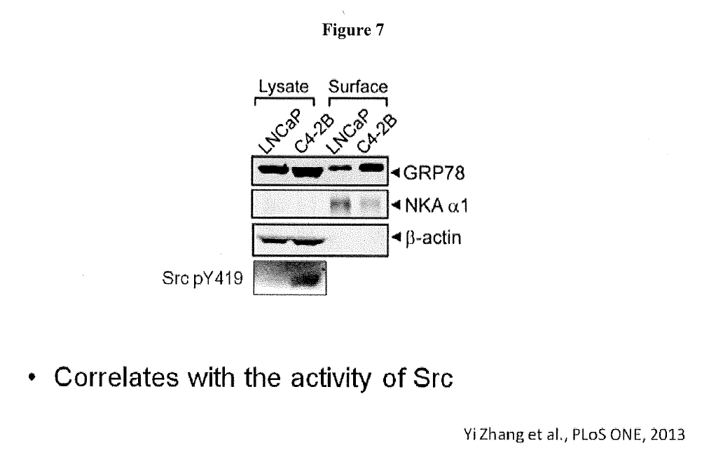

FIG. 8. Expression of ER Chaperones on the Cell Surface is Dependent of SRC Activation.

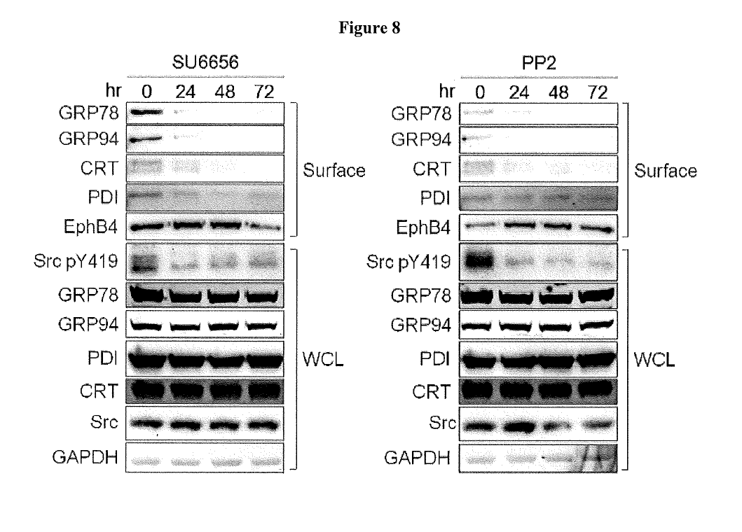

FIG. 9. Tumorigenic SRC 531 is Able to Increase Cell Surface GRP78 Level and Enhanced by ER Stress.

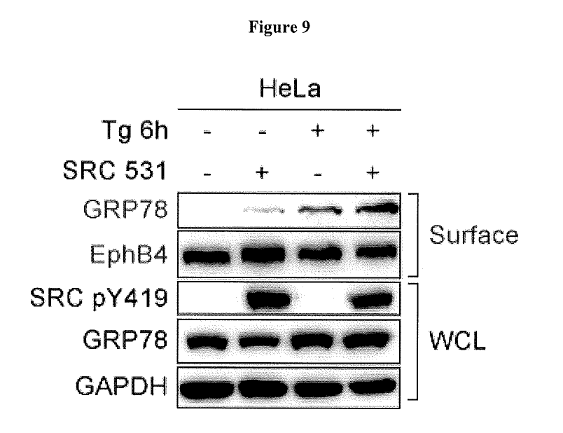

FIG. 10. Dasatinib Suppresses Cell Surface GRP78 in Multiple Myeloma. Dasatinib, a FDA approved SRC inhibitor, blocks cell surface GRP78 expression in cancer cells. (A) H929 MM cells were treated with the indicated dose of DAS and Western blotted for SRC(Y419) phosphorylation. (B) Levels of the indicated biotinylated cell surface proteins were assayed after DAS treatment (1 μM) for the indicated time.

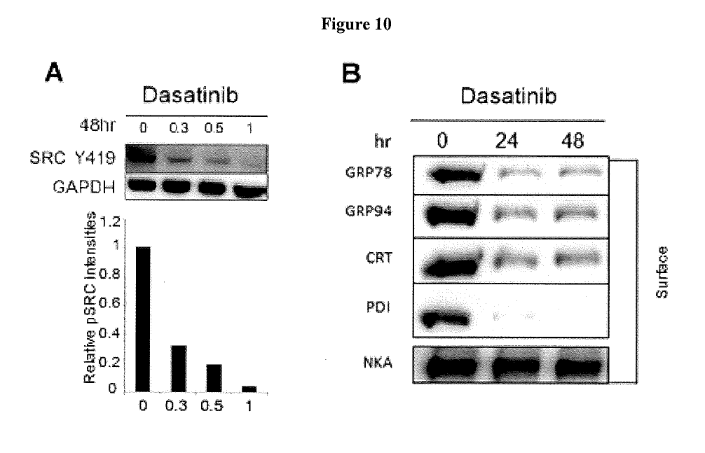

FIG. 11. Dasatinib Suppresses Cell Surface GRP78 in Multiple Myeloma. (A) Peripheral blood mononuclear cells were isolated from the bone marrow of a MM patient and sorted into CD138+ (blue) and CD138− (non-malignant) cells (orange) and csGRP78 was measured by flow cytometry, with RPMI-8226 MM cell line (red) analyzed in parallel. (B) CD138+ MM cells were treated with DAS (0.3 μM) for 24 hr and assayed for csGRP78 by flow cytometry.

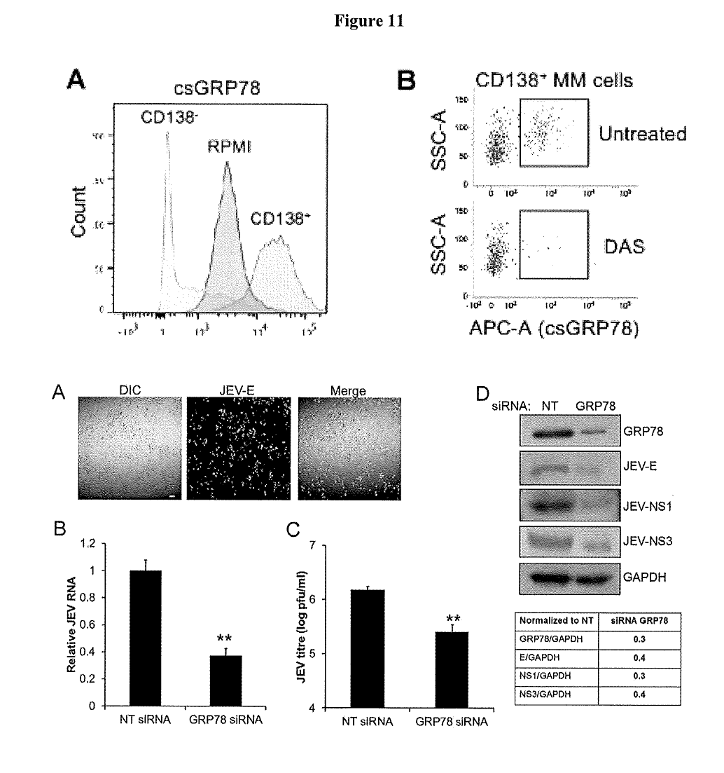

FIG. 12. Active promotion of GRP78 to the cell surface in cancer cells resistant to therapy. Representative Western blots for enhanced csGRP78 level in resistant cancer cells. Parental (P) and TamR derivatives of the human breast cancer cell models of MCF7L and MCF7/HER2-18, as well as the parental androgen sensitive LNCaP cell line and the androgen-independent C4-2B cells were subjected to biotinylation and NeutrAvidin agarose pull-down to enrich for cell surface protein. Cell surface GRP78 (csGRP78) and total intracellular GRP78 (tGRP78) in the cell lysate were probed by Western blot. The amount of total lysate was 10% of the amount used for the avidin pull-down. β-actin served as loading control for tGRP78, while membrane protein, EphB4, or Na, K-ATPase al (NKA α1) served as loading control of cell surface proteins in breast or prostate cancer cells (PCa), respectively. The experiments were repeated twice. The protein bands were quantitated and the relative levels of tGRP78 in the parental and resistant cell lines are normalized against β-actin, and csGRP78 level are normalized against EphB4 or NKA α1, respectively, which are shown by mean±standard deviation (S.D.) in the graph below. The levels in parental cell lines and in androgen sensitive cell line, LNCaP are set as 1 (15).

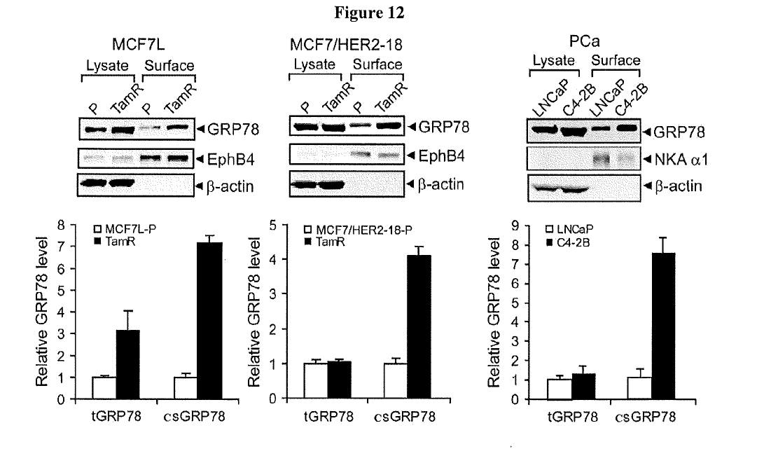

FIG. 13. ER stress further elevates csGRP78 expression level in cancer cells. (A) Parental and tamoxifen-resistant MCF7L cells were either untreated (Ctrl) or treated with 300 nM thapsigargin (Tg) for 16 h. Cell surface GRP78 were measured by FACS. Representative FACS profiles are shown and percentages of positive cells are indicated on the upper right corner. Blue dashed line, isotype control; red solid line, anti-GRP78 Ab. (B) Estrogen starvation sensitive human breast cancer cell line, MCF7/BUS, and its resistant derivative clone, MCF7/BUS-10, were either untreated (Ctrl) or treated with 10 mM 2-deoxyglucose (2DG) for 24 h. The cells were biotinylated and cell surface proteins purified by NeutrAvidin agarose pull-down. csGRP78 and tGRP78 were detected by Western Blot. β-actin served as loading control. The fold changes in csGRP78 and tGRP78 are shown below and the control condition in MCF7/BUS cells was set as 1. (C) Same as (B) except C4-2B cells treated with Tg, Tu (tunicamycin) or 2DG. NKA α1 served as cell surface protein loading control. The relative levels of tGRP78 and csGRP78 are normalized against β-actin or NKA α1, respectively, and expressed as the mean±S.D. from two independent experiments in graph (right) (15).

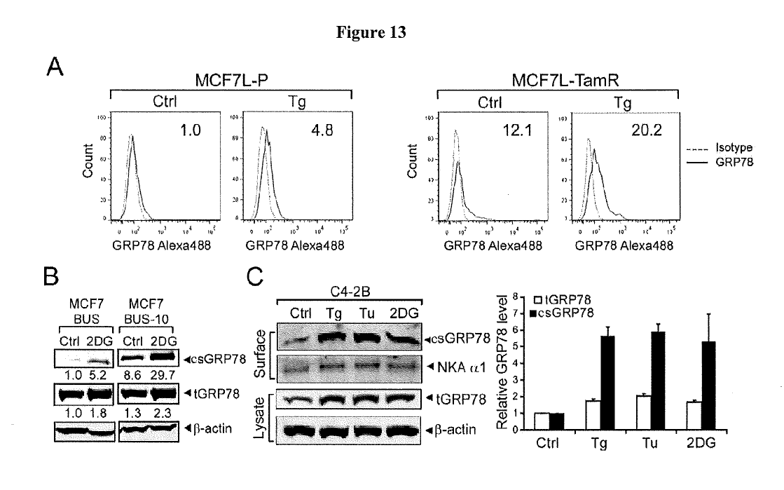

### DETAILED DESCRIPTION OF THE INVENTION

### Definitions

Unless defined otherwise, all technical and scientific terms used herein have the same meaning as is commonly understood by one of ordinary skill in the art.

As used herein, treating/treatment means any manner in which one or more of the symptoms of a disease or disorder are ameliorated or otherwise beneficially altered. Treatment also encompasses any pharmaceutical use of the compositions herein, such as use for treating a metabolic disease.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, the term “subject” refers to any animal (e.g., a mammal), including, but not limited to, humans, non-human primates, rodents, and the like, which is to be the recipient of a particular treatment. Preferably the subject is a human.

### Formulation of Pharmaceutical Compositions

The pharmaceutical compositions provided herein contain therapeutically effective amounts of one or more of compounds provided herein in a pharmaceutically acceptable carrier.

The compositions contain one or more compounds provided herein. The compounds are preferably formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation, creams, ointments and dry powder inhalers. Typically the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives is (are) mixed with a suitable pharmaceutical carrier or vehicle. The compounds may be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that treats, prevents, or ameliorates one or more of the symptoms of conditions including, but not limited to, undesired cell proliferation, cardiovascular, renal, neurodegenerative/neurologic and ophthalmic disorders, diseases or syndromes characterized by chronic inflammation and cardiovascular diseases as described herein.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated undesired cell proliferation, cardiovascular, renal, neurodegenerative/neurologic and ophthalmic disorders, diseases or syndromes characterized by chronic inflammation and cardiovascular diseases as described herein.

Typically a therapeutically effective dosage should produce a serum concentration of active ingredient of from about 0.1 ng/ml to about 50-100 μg/ml. The pharmaceutical compositions typically should provide a dosage of from about 0.001 mg to about 2000 mg of compound per kilogram of body weight per day. Pharmaceutical dosage unit forms are prepared to provide from about 1 mg to about 1000 mg and preferably from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing diseases or disorders associated with undesired cell proliferation, cardiovascular, renal, neurodegenerative/neurologic and ophthalmic disorders, diseases or syndromes characterized by chronic inflammation and cardiovascular diseases as described herein. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally, parenterally, rectally, topically and locally. For oral administration, capsules and tablets are presently preferred. The compositions are in liquid, semi-liquid or solid form and are formulated in a manner suitable for each route of administration. Preferred modes of administration include parenteral and oral modes of administration. Oral administration is presently most preferred.

Solutions or suspensions used for parenteral, intradermal, subcutaneous, or topical application can include any of the following components: a sterile diluent, such as water for injection, saline solution, polysorbate (TWEEN 80), fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of class. plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses, which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, novidone. crosnovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% active ingredient, preferably 0.1-85%, typically 75-95%.

The active compounds or pharmaceutically acceptable derivatives may be prepared with carriers that protect the compound against rapid elimination from the body, such as time release formulations or coatings.

The compositions may include other active compounds to obtain desired combinations of properties. The compounds provided herein, or pharmaceutically acceptable derivatives thereof as described herein, may also be advantageously administered for therapeutic or prophylactic purposes together with another pharmacological agent known in the general art to be of value in treating one or more of the diseases or medical conditions referred to hereinabove, such as diseases or disorders associated with undesired cell proliferation, coronary restenosis, osteoporosis, syndromes characterized by chronic inflammation, autoimmune diseases and cardiovascular diseases. It is to be understood that such combination therapy constitutes a further aspect of the compositions and methods of treatment provided herein.

One of skill in the art would recognize that other therapeutic compounds including chemotherapeutic agents, anti-inflammatory agents, and therapeutic antibodies can be used prior to, simultaneously with or following the treatments of the present invention. While not wanting to be limiting, chemotherapeutic agents include antimetabolites, such as methotrexate, DNA cross-linking agents, such as cisplatin/carboplatin; alkylating agents, such as canbusil; topoisomerase I inhibitors such as dactinomicin; microtubule inhibitors such as taxol (paclitaxol), and the like. Other chemotherapeutic agents include, for example, a vinca alkaloid, mitomycin-type antibiotic, bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methotrexate, lomustine, thioguanine, asparaginase, vinblastine, vindesine, tamoxifen, or mechlorethamine. While not wanting to be limiting, therapeutic antibodies include antibodies directed against the HER2 protein, such as trastuzumab; antibodies directed against growth factors or growth factor receptors, such as bevacizumab, which targets vascular endothelial growth factor, and OSI-774, which targets epidermal growth factor; antibodies targeting integrin receptors, such as Vitaxin (also known as MEDI-522), and the like. Classes of anticancer agents suitable for use in compositions and methods of the present invention, include, but are not limited to: 1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-16], and Teniposide [VM-26], etc.), and agents that target topoisomerase I (e.g., Camptothecin and Isirinotecan [CPT-11], etc.); 2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents [antitumor antibiotics], including, nucleic acid inhibitors (e.g., Dactinomycin [Actinomycin D], etc.), anthracyclines (e.g., Daunorubicin [Daunomycin, and Cerubidine], Doxorubicin [Adriamycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); 5) enzymes, including, L-asparaginase; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons [e.g., IFN-α, etc.] and interleukins [e.g., IL-2, etc.], etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., Batimistat, etc.); and 17) inhibitors of angiogenesis.

Examples of other therapeutic agents include the following: cyclosporins (e.g., cyclosporin A), CTLA4-Ig, antibodies such as ICAM-3, anti-IL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and/or gp39 (i.e., CD154), fusion proteins constructed from CD40 and gp39 (CD40Ig and CD8 gp39), inhibitors, such as nuclear translocation inhibitors, of NF-kappa B function, such as deoxyspergualin (DSG), cholesterol biosynthesis inhibitors such as HMG CoA reductase inhibitors (lovastatin and simvastatin), non-steroidal antiinflammatory drugs (NSAIDs) such as ibuprofen and cyclooxygenase inhibitors such as rofecoxib, steroids such as prednisone or dexamethasone, gold compounds, antiproliferative agents such as methotrexate, FK506 (tacrolimus, Prograf), mycophenolate mofetil, cytotoxic drugs such as azathioprine and cyclophosphamide, TNF-α inhibitors such as tenidap, anti-TNF antibodies or soluble TNF receptor, and rapamycin (sirolimus or Rapamune) or derivatives thereof.

Other agents that may be administered in combination with invention compositions and methods include protein therapeutic agents such as cytokines, immunomodulatory agents and antibodies. As used herein the term “cytokine” encompasses chemokines, interleukins, lymphokines, monokines, colony stimulating factors, and receptor associated proteins, and functional fragments thereof. As used herein, the term “functional fragment” refers to a polypeptide or peptide which possesses biological function or activity that is identified through a defined functional assay.

The cytokines include endothelial monocyte activating polypeptide II (EMAP-II), granulocyte-macrophage-CSF (GM-CSF), granulocyte-CSF (G-CSF), macrophage-CSF (M-CSF), IL-1, IL-2, IL-3, IL-4, IL-5, IL-6, IL-12, and IL-13, interferons, and the like and which is associated with a particular biologic, morphologic, or phenotypic alteration in a cell or cell mechanism.

### Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, preferably capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically accentable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### Injectables, Solutions and Eemulsions

Parenteral administration, generally characterized by injection, either subcutaneously, intrathecal, intrathecal, epidural, intramuscularly or intravenously is also contemplated herein. Injectables can be prepared in conventional forms, either as liquid solutions or suspensions; solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, preferably more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data.

It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg, preferably 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, preferably 5-35 mg, more preferably about 9-30 mg of lyophilized powder, is added per mL of sterile water or other suitable carrier. The precise amount depends upon the selected compound. Such amount can be empirically determined.

### Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

### Compositions for Other Routes of Administration

Other routes of administration, such as topical application, transdermal patches, and rectal administration are also contemplated herein.

For example, pharmaceutical dosage forms for rectal administration are rectal sunnositories. capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

The discovery that the 78 kDa glucose-regulated protein GRP78, traditionally regarded as a major endoplasmic reticulum (ER) chaperone and regulator of ER stress signaling, can relocalize to the cell surface under pathological stress such as cancer, changes the paradigm on how this protein exerts its pro-proliferative and anti-apoptotic function in cancer. Cell surface GRP78 (csGRP78) is emerging as a novel co-receptor controlling cell signaling, proliferation, metastasis and survival. Furthermore, since csGRP78 is preferentially expressed in cancer cells but not in normal organs, csGRP78 represents a new target for cancer-specific therapy, which are under active clinical development with therapeutic antibody entering clinical trials. csGRP78 is also required for proliferation and survival of tumor-associated endothelial cells supporting tumor growth. Furthermore, csGRP78 has been reported to be a co-receptor required for viral internalization (e.g. dengue virus, Ebola and others) and mucorales entry into host cells, initiating the infectivity and pathogenesis.

We discovered that csGRP78 is highly elevated in cancer resistant cells. We discovered that activation of the proto-oncogene tyrosine kinase protein SRC (c-SRC) is required for csGRP78 expression, as well as other ER proteins bearing the KDEL ER retention motif. Our results showed that SRC inhibitors, including Dasitinib (DAS) can block csGRP78 expression at clinically relevant dose and enhance proteasome based therapy. Thus, we have discovered a new use of an established cancer drug.

SRC is essential and sufficient for escape of KDFL-bearing ER chaperones, including GRP78, from the ER to the cell surface. Inhibition of SRC by small molecule inhibitors will impede cell surface GRP78 expression and block its pro-cancer and pro-viral entry functions. The present invention provides a new use of FDA-approved drug Dasatinib, an SRC inhibitor, as inhibitor of cell surface relocalization of csGRP78 and other KDEL-bearing ER proteins.

### GRP78 on the Surface of Proliferating Endothelial Cells

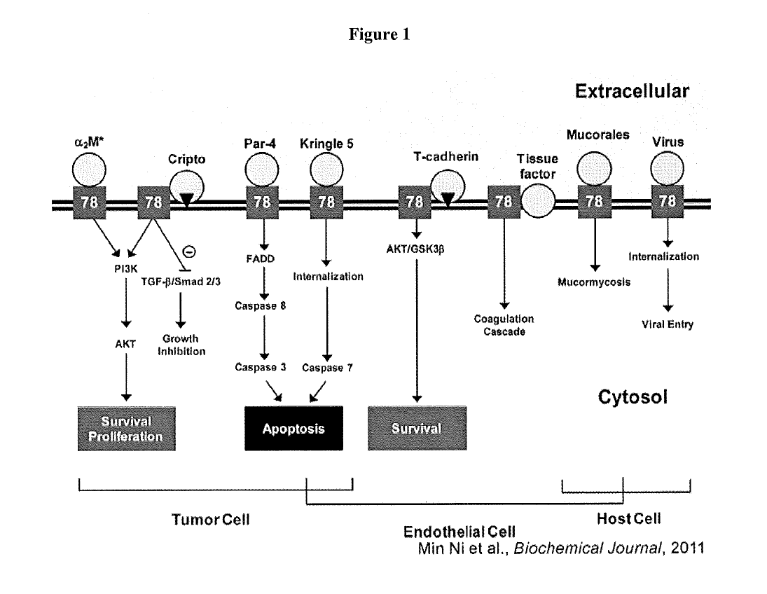

GRP78 is expressed on the cell surface of proliferating endothelial cells and monocytic cells (51, 58) (FIG. 1). GRP78 associates with major histocompatibility complex (MHC) class I on the surface of these cells and is required for MHC class I expression (59). GPI-anchored T-cadherin is reported to associate with GRP78 on the surface of vascular endothelial cells, and in this capacity, GRP78 influences endothelial cell survival as a cell surface signaling receptor (60).

As tumor progression typically requires angiogenesis for nutrient and oxygen supply, anti-angiogenic therapy exploits this requirement to block tumor growth. Kringle 5 of human plasminogen has been shown to be a binding partner of GRP78 on the surface of proliferation endothelial cells and stimulated tumor cells (58). Recombinant Kringle 5 (rK5) induces apoptosis of proliferating endothelial cells and tumor cells through binding of surface expressed GRP78 and enhancing caspase-7 activity by disruption of GRP78-procaspase-7 complex (58). Further study shows that prior irradiation significantly sensitizes the glioma microvessel endothelial cells to rK5-induced apoptosis, which required low-density lipoprotein receptor related protein 1 (LRP1) and GRP78 (61). In addition, the expression of cell surface GRP78 is elevated in VEGF-activated HUVEC cells and required for endothelial cell proliferation (62). The same study showed that cell surface GRP78 is a promising target for effective liposome drug delivery in cancer anti-neovascular therapy (62).

GRP78 was recently identified as the endothelial cell receptor required for Mucorales to penetrate and damage endothelial cells. Moreover, serum from mice vaccinated with recombinant GRP78 protected diabetic ketoacidosis mice from infection with mucoimycosis, providing a novel approach for therapeutic intervention to lethal mucoimycosis [63]. GRP78 also exists on the atherosclerosis plaque endothelial surface and negatively regulates tissue factor-mediated initiation of coagulation cascade (51). In another study, a novel peptide, RoY, was demonstrated to alleviate mouse hind limb ischemia through binding surface expressed GRP78 on hypoxic endothelial cells (64). Furthermore, another peptide derived from ADAM15, metallonrotease has also shown to activate GRP78 on endothelial cell membrane under hypoxic condition, inducing VEGF-independent angiogenesis, implying cell surface GRP78 can serve as angiogenic receptor for ischemia disease therapy (65).

SRC inhibitors will block tumor angiogenesis since cell surface GRP78 is expressed on tumor-associated endothelial cells and required for their survival and proliferation.

### Cell Surface GRP78 as Co-Receptor for Virus Internalization

Evidence is emerging that GRP78 serves as a critical portal for viral entry into host cells (FIG. 1). Previous studies on viral entry of Coxsackie virus A9 into host cells determined that it required major histocompatibility complex class I molecules. GRP78 was later found to act as co-receptor for virus internalization by associating with major histocompatibility complex class I molecules on the cell surface (66). GRP78 expressed on liver cancer cell surface acts as receptor for dengue virus serotype 2 entry and antibodies directed against both the N and C-terminus of GRP78 majorly affected the binding of the virus to the cell surface as well as the virus infectivity (67). Recently, on the study of Borna disease virus, which is characterized by highly neutropic and noncytopathic infection, GRP78 was also found on the surface of Borna disease virus targeted cells. The Borna disease virus entry was mediated by association of cell surface GRP78 with the N-terminus cleaved product of envelope glycoprotein of Borna disease virus, GP1 (68). The antibody against N-terminus of GRP78 (N20) was shown to inhibit GP1 binding to cells expressing GRP78 on cell surface and reduce virus infection.

SRC inhibitors will block viral infection into human cells since cell surface GRP78 expressed on stressed host cells serves as receptor for viral and fungal entry. This feature is particularly important in view of recent viral epidemics. Cell surface GRP78 could contribute to Ebola and Zika viral entry and infection.

### EXPERIMENTAL

Cell Culture—Human cervical cancer cell line HeLa and breast cancer cell line MCF-7 were cultured in Dulbecco's modified Eagle's medium (DMEM) containing 10% fetal bovine serum (FBS) (Life Technologies, Carlsbad, Calif.) and 1% penicillin/streptomycin. Human colon cancer cell line HCT-116 was cultured in McCoy's 5A medium containing 10% FBS and 1% penicillin/streptomycin. Human prostate cancer cell line C4-2B was cultured in RPMI 1640 medium containing 10% FBS and 1% penicillin/streptomycin. Cells were maintained at 37° C. in a humidified atmosphere of 5% CO₂ and 95% air. For stress treatment, the cells were treated with thapsigargin (Tg) at 300 nM, tunicamycin (Tu) at 1.5 μg/ml for 16 h, or 2-deoxy-D-glucose (2-DG) at 10 mM for 24 h. For Brefeldin A (BFA) treatment, the cells were incubated with 0.2 to 5 μg/ml BFA for 16 h prior to harvest. For cyclohexamide (CHX) treatment, the cells were incubated with 0.2 or 2 μg/ml CHX for 16 h. For MG-115 treatment, the cells were incubated with 20 μM for 16 h prior to harvest. All the agents mentioned above were purchased from Sigma-Aldrich, St. Louis, Mo.

Transfection and Collection of Secreted Proteins—Transfection was performed as described using BioT (Bioland Scientific, Paramount, Calif.) following the manufacturer's instructions (9). The secreted proteins were collected as described (9).

Cell Surface Protein Biotinylation and Avidin Pull-down—Following treatment, the cells were washed with cold PBS 3 times. EZ-Link Sulfo-NHS-SS-Biotin (Thermo Scientific, Waltham, Mass.) in PBS at 0.5 mg/ml was added and the cells were gently shaken at 4° C. for 30 min. To stop the biotinylation reaction, the biotin solution was removed, and the cells were rinsed three times with the quenching buffer containing Tris-Cl pH 7.4 in cold PBS. Then cells were rinsed with cold PBS and subjected to either sodium carbonate extraction (see below) or radioimmune precipitation (RIPA) lysis. The RIPA lysis buffer was supplemented with protease and phosphatase inhibitor cocktail (Thermo Scientific). Protein concentrations were determined by Bradford assay (Bio-Rad Lab, Hercules, Calif.). Part of the lysate was saved as whole cell lysate for Western blots to measure the total level of the target proteins. To purify the surface proteins, the remaining lysates were mixed with High Capacity NeutrAvidin Agarose Resin (Thermo Scientific) at room temperature for 1 h, and the resin was washed by RIPA buffer and centrifuged at 3,000× g for 1 min ten times. The cell surface proteins were released by the addition of 50 μl of 2× SDS-PAGE sample buffer, followed by heating at 95° C. for 5 min, and centrifuged at 6,000× g for 5 min to collect the supernatant.

Immunoblot Analysis—Protein samples were subjected to 10% or 15% SDS-PAGE and Western blot analysis as previously described (9). Primary antibodies used are as follows: anti-ANXA2 mouse antibody (610068, BD Biosciences, San Jose, Calif.), 1:2500; anti-β-actin mouse antibody (A5316, Sigma-Aldrich), 1:5000; anti-Calreticulin rabbit antibody (catalogue no. 2891, Cell Signaling, Danvers, Mass.), 1:5000; anti-Calnexin rabbit antibody (ADI-SPA-860, Enzo Life Sciences), 1:1000; anti-EphB4 mouse antibody (MAb131) (22), 1:1000; anti-FLAG M2 mouse antibody (F1804, Sigma-Aldrich), 1:1000; anti-GAPDH mouse antibody (sc-32233, Santa Cruz Biotechnology, Inc., Dallas, Tex.), 1:1000; anti-GRP78 mouse antibody (MAb159) (16), 1:1000; anti-GRP78 rat antibody (sc-13539, Santa Cruz Biotechnology, Inc.), 1:1000; anti-GRP94 rat antibody (SPA-851, Enzo Life Sciences), 1:1000; anti-HA rabbit antibody (sc-805, Santa Cruz Biotechnology, Inc.), 1:1000; anti-HSP70 mouse antibody (sc-66048, Santa Cruz Biotechnology, Inc.) 1:500; anti-HTJ1 rabbit antibody (GTX103858, GeneTex, Inc., Irvine, Calif.) 1:500; anti-Integrin β1 rabbit antibody (EP1041Y, Millipore, Billerica, Mass.), 1:500; anti-PDI rabbit antibody (ADI-SPA-890, Enzo Life Sciences), 1:1000; anti-PDI rabbit antibody (sc-20132, Santa Cruz Biotechnology, Inc.), 1:500; anti-uPAR rabbit antibody (GTX100467, GeneTex, Inc.), 1:500. The secondary antibodies used in this study are as follows: horseradish peroxidase conjugate goat anti-mouse, anti-rabbit, and anti-rat antibodies (sc-2005, sc-2004, sc-2006, Santa Cruz Biotechnology, Inc.), and goat anti-mouse IRDye® 800CW, 1:7500, and goat anti-rabbit IRDye® 680RD secondary antibodies (LI-COR Biosciences, Lincoln, Nebr.) 1:7500. The experiments were repeated 2-4 times. Protein levels were visualized and quantitated by ChemiDoc™ XRS+ Imager (Bio-Rad Lab) or LI-COR Odyssey (LI-COR Biosciences).

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

The following references are each relied upon and incorporated herein in their entirety.
1. Ni, M., and Lee, A. S. (2007) ER chaperones in mammalian development and human diseases. FEBS Lett. 581, 3641-3651
2. Lee, A. S. (2014) Glucose-regulated proteins in cancer: molecular mechanisms and therapeutic potential. Nat. Rev. Cancer 14, 263-276
3. Munro, S., and Pelham, H. R. (1986) An Hsp70-like protein in the ER: identity with the 78 kd glucose-regulated protein and immunoglobulin heavy chain binding protein. Cell 46, 291-300
4. Luo, B., and Lee, A. S. (2013) The critical roles of endoplasmic reticulum chaperones and unfolded protein response in tumorigenesis and anticancer therapies. Oncogene 32, 805-818
5. Bertolotti. A., Zhang, Y., Hendershot, L. M., Harding, H. P., and Ron, D. (2000) Dynamic interaction of BiP and ER stress transducers in the unfolded-protein response. Nat. Cell Biol. 2, 326-332
6. Ni, M., Zhang, Y, and Lee, A. S. (2011) Beyond the endoplasmic reticulum: atypical GRP78 in cell viability, signalling and therapeutic targeting. Biochem. J 434, 181-188
7. Arap, M. A., Landenranta, J., Mintz, P. J., Hajitou, A., Sarkis, A. S., Arap, W., and Pasqualini, R. (2004) Cell surface expression of the stress response chaperone GRP78 enables tumor targeting by circulating ligands. Cancer Cell 6, 275-284
8. Gonzalez-Gronow, M., Selim, M. A., Papalas, J., and Pizzo, S. V. (2009) GRP78: a multifunctional receptor on the cell surface. Antioxid. Redox Signal. 11, 2299-2306
9. Zhang, Y., Liu, R., Ni, M., Gill, P., and Lee, A. S. (2010) Cell surface relocalization of the endoplasmic reticulum chaperone and unfolded protein response regulator GRP78/BiP. J. Biol. Chem. 285, 15065-15075
10. Misra, U. K., Gonzalez-Gronow, M., Gawdi, G, Hart, J. P., Johnson, C. E., and Pizzo, S. V. (2002) The role of Grp 78 in alpha 2-macroglobulin-induced signal transduction. Evidence from RNA interference that the low density lipoprotein receptor-related protein is associated with, but not necessary for, GRP 78-mediated signal transduction. J Biol. Chem. 277, 42082-42087
11. Shani, G, Fischer, W. H., Justice, N. J., Kelber, J. A., Vale, W., and Gray, P. C. (2008) GRP78 and Cripto form a complex at the cell surface and collaborate to inhibit transforming growth factor beta signaling and enhance cell growth. Mol. Cell. Biol. 28, 666-677
12. Spike, B. T., Kelber, J. A., Booker, E., Kalathur, M., Rodewald, R., Lipianskaya, J., La, J., He, M., Wright, T., Klemke, R., Wahl, G. M., and Gray, P. C. (2014) CRIPTO/GRP78 signaling maintains fetal and adult mammary stem cells ex vivo. Stem Cell Reports 2, 427-439
13. Miao, Y R., Eckhardt, B. L., Cao, Y, Pasqualini, R., Argani, P., Arap, W., Ramsay, R. G, and Anderson, R. L. (2013) Inhibition of established micrometastases by targeted drug delivery via cell surface-associated GRP78. Clin. Cancer Res. 19, 2107-2116
14. Rasche, L., Duell, J., Morgner, C., Chatterjee, M., Hensel, F., Rosenwald, A., Einsele, H., Topp, M. S., and Brandlein, S. (2013) The natural human IgM antibody PAT-SM6 induces apoptosis in primary human multiple myeloma cells by targeting heat shock protein GRP78. PLoS One 8, e63414
15. Zhang, Y, Tseng, C. C., Tsai, Y. L., Fu, X., Schiff, R., and Lee, A. (2013) Cancer cells resistant to therapy promote cell surface relocalization of GRP78 which complexes with PI3K and enhances PI(3,4,5)P3 production. PLoS One 8, e80071
16. Liu, R., Li, X., Gao, W., Zhou, Y., Wey, S., Mitra, S. K., Krasnoperov, V., Dong, D., Liu, S., Li, D., Zhu, G, Louie, S., Conti, P. S., Li, Z., Lee, A. S., and Gill, P. S. (2013) Monoclonal antibody against cell surface GRP78 as a novel agent in suppressing PI3K/AKT signaling, tumor growth and metastasis. Clin. Cancer Res. 19, 6802-6811
17. de Ridder, G G, Ray, R., and Pizzo, S. V. (2012) A murine monoclonal antibody directed against the carboxyl-terminal domain of GRP78 suppresses melanoma growth in mice. Melanoma Res. 22, 225-235
18. Sato, M., Yao, V. J., Arap, W., and Pasqualini, R. (2010) GRP78 signaling hub a receptor for targeted tumor therapy. Adv. Genet. 69, 97-114
19. Shin, B. K., Wang, H., Yim, A. M., Le Naour, F., Brichory, F., Jang, J. H., Zhao, R., Purays, E., Tra, J., Michael, C. W., Misek, D. E., and Hanash, S. M. (2003) Global profiling of the cell surface proteome of cancer cells uncovers an abundance of proteins with chaperone function. J. Biol. Chem. 278, 7607-7616
20. Pinaud, F., and Dahan, M. (2011) Targeting and imaging single biomolecules in living cells by complementation-activated light microscopy with split-fluorescent proteins. Proc. Natl. Acad. Sci. USA 108, E201-210
21. Okamoto, T., Schwab, R. B., Scherer, P. E., and Lisanti, M. P. (2001) Analysis of the association of proteins with membranes. Curr. Protoc. Cell Biol. Chapter 5, 5:5.4:5.4.1-5.4.17
22. Liu, R., Ferguson, B. D., Zhou, Y., Naga, K., Salgia, R., Gill, P. S., and Krasnoperov, V. (2013) EphB4 as a therapeutic target in mesothelioma. BMC Cancer 13, 269
23. Fu, Y., Li, J., and Lee, A. S. (2007) GRP78/BiP inhibits endoplasmic reticulum BIK and protects human breast cancer cells against estrogen-starvation induced apoptosis. Cancer Res. 67, 3734-3740
24. Ni, M., Zhou, H., Wey, S., Baumeister, P., and Lee, A. S. (2009) Regulation of PERK signaling and leukemic cell survival by a novel cytosolic isoform of the UPR regulator GRP78/BiP. PLoS One 4, e6868
25. Tamilselvam, B., and Daefler, S. (2008) Francisella targets cholesterol-rich host cell membrane domains for entry into macrophages. J. Immunol. 180, 8262-8271
26. Heilemann, M., van de Linde, S., Schuttpelz, M., Kasper, R., Seefeldt, B., Mukherjee, A., Tinnefeld, P., and Sauer, M. (2008) Subdiffraction-resolution fluorescence imaging with conventional fluorescent probes. Angew. Chem. Int. Ed. Engl. 47, 6172-6176
27. Huang, B., Wang, W., Bates, M., and Zhuang, X. (2008) Three-dimensional super-resolution imaging by stochastic optical reconstruction microscopy. Science 319, 810-813
28. Dempsey, G T., Vaughan, J. C., Chen, K. H., Bates, M., and Zhuang, X. (2011) Evaluation of fluorophores for optimal performance in localization-based super-resolution imaging. Nat. Methods 8, 1027-1036
29. Wolter, S., Loschberger, A., Holm, T., Aufmkolk, S., Dabauvalle, M. C., van de Linde, S., and Sauer, M. (2012) rapidSTORM: accurate, fast open-source software for localization microscopy. Nat. Methods 9, 1040-1041
30. Pettersen, E. F., Goddard, T. D., Huang, C. C., Couch, G S., Greenblatt, D. M., Meng, E. C., and Ferrin, T. E. (2004) UCSF Chimera—a visualization system for exploratory research and analysis. J Comput. Chem. 25, 1605-1612
31. Reddy, R. K., Mao, C., Baumeister, P., Austin, R. C., Kaufman, R. J., and Lee, A. S. (2003) Endoplasmic reticulum chaperone protein GRP78 protects cells from apoptosis induced by topoisomerase inhibitors: role of ATP binding site in suppression of caspase-7 activation. J Biol. Chem. 278, 20915-20924
32. Rao, R. V., Peel, A., Logvinova, A., del Rio, G., Hermel, E., Yokota, T., Goldsmith, P. C., Ellerby, L. M., Ellerby, H. M., and Bredesen, D. E. (2002) Coupling endoplasmic reticulum stress to the cell death program: role of the ER chaperone GRP78. FEBS Lett. 514, 122-128
33. Ellgaard, L., and Helenius, A. (2001) ER quality control: towards an understanding at the molecular level. Curr Opin. Cell Biol. 13, 431-437
34. Flanagan, J. G, and Vanderhaeghen, P. (1998) The ephrins and Eph receptors in neural development. Annu. Rev. Neurosci. 21, 309-345
35. Gerke, V., Creutz, C. E., and Moss, S. E. (2005) Annexins: linking Ca2+ signalling to membrane dynamics. Nat. Rev. Mol. Cell Biol. 6, 449-461
36. Satpute-Krishnan, P., Ajinkya, M., Bhat, S., Itakura, E., Hegde, R. S., and Lippincott-Schwartz, J. (2014) ER stress-induced clearance of misfolded GPI-anchored proteins via the secretory pathway. Cell 158, 522-533
37. Klausner, R. D., Donaldson, J. G, and Lippincott-Schwartz, J. (1992) Brefeldin A: insights into the control of membrane traffic and organelle structure. J. Cell Biol. 116, 1071-1080
38. Liu, E. S., Ou, J. H., and Lee, A. S. (1992) Brefeldin A as a regulator of grp78 gene expression in mammalian cells. J. Biol. Chem. 267, 7128-7133
39. Deora, A. B., Kreitzer, G, Jacovina, A. T., and Hajjar, K. A. (2004) An annexin 2 phosphorylation switch mediates p11-dependent translocation of annexin 2 to the cell surface. J. Biol. Chem. 279, 43411-43418
40. Afshar, N., Black, B. E., and Paschal, B. M. (2005) Retrotranslocation of the chaperone calreticulin from the endoplasmic reticulum lumen to the cytosol. Mol. Cell. Biol. 25, 8844-8853
41. Awad, W., Estrada, I., Shen, Y., and Hendershot, L. M. (2008) BiP mutants that are unable to interact with endoplasmic reticulum DnaJ proteins provide insights into interdomain interactions in BiP. Proc. Natl. Acad. Sci. USA 105, 1164-1169
42. Wei, J., Gaut, J. R., and Hendershot, L. M. (1995) In vitro dissociation of BiP-peptide complexes requires a conformational change in BiP after ATP binding but does not require ATP hydrolysis. J. Biol. Chem. 270, 26677-26682
43. Guo, F., and Snapp, E. L. (2013) ERdj3 regulates BiP occupancy in living cells. J. Cell Sci. 126, 1429-1439
44. Misra, U. K., Gonzalez-Gronow, M., Gawdi, G, and Pizzo, S. V. (2005) The role of MTJ-1 in cell surface translocation of GRP78, a receptor for alpha 2-macroglobulin-dependent signaling. J. Immnol. 174, 2092-2097
45. Nakayama, H., Fukuda, S., Inoue, H., Nishida-Fukuda, H., Shirakata, Y, Hashimoto, K., and Higashiyama, S. (2012) Cell surface annexins regulate ADAM-mediated ectodomain shedding of proamphiregulin. Mol. Biol. Cell 23, 1964-1975
46. Mahalka, A. K., Kirkegaard, T., Jukola, L. T., Jaattela, M., and Kinnunen, P. K. (2014) Human heat shock protein 70 (Hsp70) as a peripheral membrane protein. Biochim. Biophys. Acta 1838, 1344-1361
47. Rothberg, K. G, Heuser, J. E., Donzell, W. C., Ying, Y. S., Glenney, J. R., and Anderson, R. G. (1992) Caveolin, a protein component of caveolae membrane coats. Cell 68, 673-682
48. Patlolla, J. M., Swamy, M. V., Raju, J., and Rao, C. V. (2004) Overexpression of caveolin-1 in experimental colon adenocarcinomas and human colon cancer cell lines. Oncol. Rep. 11, 957-963
49. Obeid, M., Panaretakis, T., Tesniere, A., Joza, N., Tufi, R., Apetoh, L., Ghiringhelli, F., Zitvogel, L., and Kroemer, G. (2007) Leveraging the immune system during chemotherapy: moving calreticulin to the cell surface converts apoptotic death from “silent” to immunogenic. Cancer Res. 67, 7941-7944
50. Bellani, S., Mescola, A., Ronzitti, G, Tsushima, H., Tilve, S., Canale, C., Valtorta, F., and Chieregatti, E. (2014) GRP78 clustering at the cell surface of neurons transduces the action of exogenous alpha-synuclein. Cell Death Differ., in press, 10.1038/cdd.2014.1111
51. Bhattacharjee, G, Ahamed, J., Pedersen, B., El-Sheikh, A., Mackman, N., Ruf, W., Liu, C., and Edgington, T. S. (2005) Regulation of tissue factor—mediated initiation of the coagulation cascade by cell surface grp78. Arterioscler. Thromb. Vasc. Biol. 25, 1737-1743
52. Al-Hashimi, A. A., Caldwell, J., Gonzalez-Gronow, M., Pizzo, S. V., Aboumrad, D., Pozza, L., Al-Bayati, H., Weitz, J. I., Stafford, A., Chan, H., Kapoor, A., Jacobsen, D. W., Dickhout, J. G., and Austin, R. C. (2010) Binding of anti-GRP78 autoantibodies to cell surface GRP78 increases tissue factor procoagulant activity via the release of calcium from endoplasmic reticulum stores. J. Biol. Chem. 285, 28912-28923
53. Ray, R., de Ridder, G G, Eu, J. P., Paton, A. W., Paton, J. C., and Pizzo, S. V. (2012) The Escherichia coli subtilase cytotoxin A subunit specifically cleaves cell-surface GRP78 protein and abolishes COOH-terminal-dependent signaling. J. Biol. Chem. 287, 32755-32769
54. Gray, P. C., and Vale, W. (2012) Cripto/GRP78 modulation of the TGF-beta pathway in development and oncogenesis. FEBS Lett 586, 1836-1845
55. Burikhanov, R., Zhao, Y., Goswami, A., Qiu, S., Schwarze, S. R., and Rangnekar, V. M. (2009) The tumor suppressor Par-4 activates an extrinsic pathway for apoptosis. Cell 138, 377-388
56. Kepes, F. (1996) The “+70 pause”: hypothesis of a translational control of membrane protein assembly. J. Mol. Biol. 262, 77-86
57. Wu, M. M., Buchanan, J., Luik, R. M., and Lewis, R. S. (2006) Ca2+ store depletion causes STIM1 to accumulate in ER regions closely associated with the plasma membrane. J. Cell Biol. 174, 803-813
58. Davidson, D. J., Haskell, C., Majest, S., Kherzai, A., Egan, D. A., Walter, K. A., Schneider, A., Gubbins, E. F., Solomon, L., Chen, Z., Lesniewski, R. and Henkin, J. (2005) Kringle 5 of human plasminogen induces apoptosis of endothelial and tumor cells through surface-expressed glucose-regulated protein 78. Cancer Res. 65, 4663-4672
59. Triantafilou, M., Fradelizi, D. and Triantafilou, K. (2001) Major histocompatibility class one molecule associates with glucose regulated protein (GRP) 78 on the cell surface. Hum. Immunol. 62, 764-770
60. Philippova, M., Ivanov, D., Joshi, M. B., Kyriakakis, E., Rupp, K., Afonyushkin, T., Bochkov, V., Erne, P. and Resink, T. J. (2008) Identification of proteins associating with glycosylphosphatidylinositol-anchored T-cadherin on the surface of vascular endothelial cells: role for Grp78/BiP in T-cadherin-dependent cell survival. Mol. Cell. Biol. 28, 4004-4017
61. McFarland, B. C., Stewart, J., Jr., Hamza, A., Nordal, R., Davidson, D. J., Henkin, J. and Gladson, C. L. (2009) Plasminogen Kringle 5 induces apoptosis of brain microvessel endothelial cells: sensitization by radiation and requirement for GRP78 and LRP1. Cancer Res. 69, 5537-5545
62. Katanasaka, Y., Ishii, T., Asai, T., Naitou, H., Maeda, N., Koizumi, F., Miyagawa, S., Ohashi, N. and Oku, N. (2010) Cancer antineovascular therapy with liposome drug delivery systems targeted to BiP/GRP78. Int. J. Cancer 127, 2685-2698
63. Liu, M., Spellberg, B., Phan, Q. T., Fu, Y., Lee, A. S., Edwards, J. E., Jr., Filler, S. G. and Ibrahim, A. S. (2010) The endothelial cell receptor GRP78 is required for mucormycosis pathogenesis in diabetic mice. J. Clin. Invest. 120, 1914-1924
64. Hardy, B., Battler, A., Weiss, C., Kudasi, O. and Raiter, A. (2008) Therapeutic angiogenesis of mouse hind limb ischemia by novel peptide activating GRP78 receptor on endothelial cells. Biochem. Pharmacol. 75, 891-899
65. Raiter, A., Weiss, C., Bechor, Z., Ben-Dor, I., Battler, A., Kaplan, B. and Hardy, B. (2010) Activation of GRP78 on endothelial cell membranes by an ADAM15-derived peptide Induces angiogenesis. J. Vasc. Res. 47, 399-411
66. Triantafilou, K., Fradelizi, D., Wilson, K. and Triantafilou, M. (2002) GRP78, a coreceptor for coxsackievirus A9, interacts with major histocompatibility complex class I molecules which mediate virus internalization. J. Virol. 76, 633-643
67. Jindadamrongwech, S., Thepparit, C. and Smith, D. R. (2004) Identification of GRP 78 (BiP) as a liver cell expressed receptor element for dengue virus serotype 2. Arch. Virol. 149, 915-927
68. Honda, T., Horie, M., Daito, T., Ikuta, K. and Tomonaga, K. (2009) Molecular chaperone BiP interacts with Borna disease virus glycoprotein at the cell surface. J. Virol. 83, 12622-12625
69. Nain, M., et al., (2017) GRP78 is an Important Host Factor for Japanese Encephalitis Virus Entry and Replication in Mammalian Cells. J. Virol. 91, 1-21
70. Tsai, Y. L., et al. (2015) Characterization and Mechanism of Stress-Induced Translocation of 78-Kilodalton Glucose-regulated Protein (GRP78) to the Cell Surface. J. Biol. Chem. 290, 8049-8063
71. Min, N., et al. (2011) Beyond the Endoplasmic Reticulum: Atypical GRP78 in Cell Viability, Signalling and Therapeutic Targeting. Biochem. J 434, 181-188
72. Martins, M., et al. (2012) Listeria monocytogenes Triggers the Cell Surface Expression of Gp96 Protein and Interacts with Its N Terminus to Support Cellular Infection. J. Biol. Chem. 287, 43083-43093

What is claimed is:

1. A method of treating cancer, comprising:
administering to a subject in need thereof an effective amount of a pharmaceutical composition comprising a compound that inhibits proto-oncogene tyrosine kinase protein SRC (c-SRC), wherein the inhibition of SRC blocks cell surface glucose-regulated protein GRP78 and/or other endoplasmic reticulum ER luminal proteins dependent on SRC from going to the cell surface.

2. The method of claim 1, wherein the compound that inhibits SRC is selected from the group consisting of dasatinib, saracatinib, bosutinib and KX-01.

3. The method of claim 2, wherein the compound that inhibits SRC is dasatinib.

4. The method of claim 1, wherein the cancer is selected from the group consisting of bladder cancer, breast cancer, ovarian cancer, pancreatic cancer, and gastric cancer, cervical cancer, colon cancer, endometrial cancer, head and neck cancer, lung cancer, melanoma, multiple myeloma, leukemia, non-hodgkin's lymphoma, prostate cancer, rectal cancer, malignant melanomas, alimentary/gastrointestinal tract cancer, liver cancer, skin cancer, lymphoma, kidney cancer, muscle cancer, bone cancer, brain cancer, eye or ocular cancer, rectal cancer, colon cancer, cervical cancer, bladder cancer, oral cancer, benign and malignant tumors, stomach cancer, corpus uteri, testicular cancer, renal cancer, throat cancer, acute lymphocytic leukemia, acute myelogenous leukemia, Ewing's Sarcoma, Kaposi's Sarcoma, basal cell carinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, neurofibromatosis, tuberous sclerosis, hemangiomas, and lymphangiogenesis.

5. The method of claim 4, wherein the cancer is multiple myeloma.

6. A method of treating multiple myeloma, comprising:
administering to a subject in need thereof an effective amount of a pharmaceutical composition comprising dasatinib.

7. A method of hindering or preventing tumor angiogenesis, comprising:
administering to a subject in need thereof an effective amount of a pharmaceutical composition comprising a compound that inhibits proto-oncogene tyrosine kinase protein SRC (c-SRC), wherein the inhibition of SRC blocks cell surface glucose-regulated protein GRP78 and/or other endoplasmic reticulum ER luminal proteins dependent on SRC from going to the cell surface.

8. The method of claim 7, wherein the compound that inhibits SRC is selected from the group consisting of dasatinib, saracatinib, bosutinib and KX-01.

9. The method of claim 8, wherein the compound that inhibits SRC is dasatinib.

10. A method of hindering or preventing viral infection into human cells, comprising:
administering to a subject in need thereof an effective amount of a pharmaceutical composition comprising a compound that inhibits proto-oncogene tyrosine kinase protein SRC (c-SRC), wherein the inhibition of SRC blocks cell surface glucose-regulated protein GRP78 and/or other endoplasmic reticulum ER luminal proteins dependent on SRC from going to the cell surface.

11. The method of claim 10, wherein the compound that inhibits SRC is selected from the group consisting of dasatinib, saracatinib, bosutinib and KX-01.

12. The method of claim 11, wherein the compound that inhibits SRC is dasatinib.

13. A method of hindering or preventing fungal entry into host cells, comprising:
administering to a subject in need thereof an effective amount of a pharmaceutical composition comprising a compound that inhibits proto-oncogene tyrosine kinase protein SRC (c-SRC), wherein the inhibition of SRC blocks cell surface glucose-regulated protein GRP78 and/or other endoplasmic reticulum ER luminal proteins dependent on SRC from going to the cell surface.

14. The method of claim 13, wherein the compound that inhibits SRC is selected from the group consisting of dasatinib, saracatinib, bosutinib and KX-01.

15. The method of claim 14, wherein the compound that inhibits SRC is dasatinib.

16. A method of hindering or preventing Listeria monocytogenes infection into human cells, comprising:
administering to a subject in need thereof an effective amount of a pharmaceutical composition comprising a compound that inhibits proto-oncogene tyrosine kinase protein SRC (c-SRC), wherein the inhibition of SRC blocks cell surface glucose-regulated protein GRP78 and/or other endoplasmic reticulum ER luminal proteins dependent on SRC from going to the cell surface.

17. The method of claim 16, wherein the compound that inhibits SRC is selected from the group consisting of dasatinib, saracatinib, bosutinib and KX-01.

18. The method of claim 17, wherein the compound that inhibits SRC is dasatinib.

19. A method of reducing drug resistance in cancer cells, comprising:
administering to a subject in need thereof an effective amount of a pharmaceutical composition comprising a compound that inhibits proto-oncogene tyrosine kinase protein SRC (c-SRC), wherein the inhibition of SRC blocks cell surface glucose-regulated protein GRP78 and/or other endoplasmic reticulum ER luminal proteins dependent on SRC from going to the cell surface.

20. The method of claim 19, wherein the compound that inhibits SRC is selected from the group consisting of dasatinib, saracatinib, bosutinib and KX-01.

21. The method of claim 20, wherein the compound that inhibits SRC is dasatinib.

22. A method of hindering or preventing viral entry into host cells, comprising:
administering to a subject in need thereof an effective amount of a pharmaceutical composition comprising a compound that inhibits proto-oncogene tyrosine kinase protein SRC (c-SRC), wherein the inhibition of SRC blocks cell surface glucose-regulated protein GRP78 and/or other endoplasmic reticulum ER luminal proteins dependent on SRC from going to the cell surface.

23. The method of claim 22, wherein the compound that inhibits SRC is selected from the group consisting of dasatinib, saracatinib, bosutinib and KX-01.

24. The method of claim 23, wherein the compound that inhibits SRC is dasatinib.

25. The method of claim 1, further comprising concurrently or sequentially administering to the subject one or more additional treatments selected from the group consisting of chemotherapy, immune and radiation therapy.

26. The method of claim 1, wherein the pharmaceutical composition further comprises a therapeutic antibody, an antibody-drug conjugate, a radioimmunotherapy agent, a small molecule therapeutic agent or an immune stimulating agent.

27. The method of claim 6, wherein the pharmaceutical composition further comprises a therapeutic antibody, an antibody-drug conjugate, a radioimmunotherapy agent, a small molecule therapeutic agent or an immune stimulating agent.

28. The method of claim 7, wherein the pharmaceutical composition further comprises a therapeutic antibody, an antibody-drug conjugate, a radioimmunotherapy agent, a small molecule therapeutic agent or an immune stimulating agent.

29. The method of claim 10, wherein the pharmaceutical composition further comprises a therapeutic antibody, an antibody-drug conjugate, a radioimmunotherapy agent, a small molecule therapeutic agent or an immune stimulating agent.

30. The method of claim 13, wherein the pharmaceutical composition further comprises a therapeutic antibody, an antibody-drug conjugate, a radioimmunotherapy agent, a small molecule therapeutic agent or an immune stimulating agent.

31. The method of claim 16, wherein the pharmaceutical composition further comprises a therapeutic antibody, an antibody-drug conjugate, a radioimmunotherapy agent, a small molecule therapeutic agent or an immune stimulating agent.

32. The method of claim 19, wherein the pharmaceutical composition further comprises a therapeutic antibody, an antibody-drug conjugate, a radioimmunotherapy agent, a small molecule therapeutic agent or an immune stimulating agent.

33. The method of claim 22, wherein the pharmaceutical composition further comprises a therapeutic antibody, an antibody-drug conjugate, a radioimmunotherapy agent, a small molecule therapeutic agent or an immune stimulating agent.
